(19) United States
(12) Patent Application Publication
(10) Pub. No.: US 2023/0190702 A1
(43) Pub. Date: Jun. 22, 2023

(54) A COMPOSITION FOR IMPROVING QOL OF A 40-YEAR-OLD OR OLDER POSTMENOPAUSAL WOMAN WITHOUT MENOPAUSAL DISORDER

(71) Applicant: DAICEL CORPORATION, Osaka-shi, Osaka (JP)

(72) Inventors: Kenichi OE, Tokyo (JP); Yuichi UKAWA, Tokyo (JP)

(73) Assignee: DAICEL CORPORATION, Osaka-shi, Osaka (JP)

(21) Appl. No.: 17/926,424

(22) PCT Filed: May 11, 2021
(86) PCT No.: PCT/JP2021/017946

§ 371 Date: Nov. 18, 2022

(30) Foreign Application Priority Data
May 21, 2020 (JP) 2020-088810

(51) Int. Cl. A61K 31/353; A23L 33/105; A61P 5/30

(52) U.S. Cl. CPC A61K 31/353; A23L 33/105; A61P 5/30

(57) ABSTRACT

An object of the present disclosure is at least to provide a technique for improving QOL of a 40-year-old or older postmenopausal woman without menopausal disorder, and the object is achieved by a composition for improving QOL of a 40-year-old or older postmenopausal woman without menopausal disorder, comprising equols.

### TECHNICAL FIELD

The present disclosure relates to a composition for improving QOL of a 40-year-old or older postmenopausal woman without menopausal disorder.

### BACKGROUND ART

Equol is known to have an ameliorating effect on the menopause symptoms (e.g., stiff shoulder, hot flush) or osteoporosis (Non-Patent Documents 1 and 2). Further, equol is known to have an ameliorating effect on the female-specific physical and mental unpleasant symptoms (premenstrual syndrome (PMS), pain, low mood, changes in concentration ability and behavior) (Patent Document 1).

Meanwhile, it is not known that the quality of life (QOL) is improved when equol is ingested by a person other than the persons who have the menopause symptoms or the female-specific physical and mental unpleasant symptoms.

### PRIOR ART DOCUMENTS

### Patent Documents

[Patent Document 1] WO 2017/154865

### Non-Patent Documents

[Non-Patent Document 1] Menopause, Vol. 16, No. 1, p. 141-8 (2009) [Non-Patent Document 2] Menopause, Vol. 18, No. 5, p. 563-74 (2011)

### SUMMARY OF THE INVENTION

### Problems to be Solved by the Invention

An object of the present disclosure is at least to provide a technique for improving the QOL of a 40-year-old or older postmenopausal woman without menopausal disorder.

### Means for Solving the Problems

(1) A composition for improving QOL of a 40-year-old or older postmenopausal woman without menopausal disorder, comprising equols.

(2) The composition according to (1), wherein the QOL improvement is an improvement in physical symptom or an improvement in mental symptom.

(3) The composition according to (2), wherein the improvement in physical symptom is an improvement in eye fatigue, an improvement in physical pain, an improvement in cardiac ischemia, an improvement in fatigue, an improvement in dry mouth, an improvement in skin condition, an improvement in bronchitis, an improvement in gray hair, an improvement in autonomic nerve balance, an improvement in hearing loss, an improvement in swelling, an improvement in frequent urination, an improvement in insomnia, a maintenance of body weight, a gastric regulation, an improvement of immunity, an intestinal regulation, or hair growth.

(4) The composition according to (2), wherein the improvement in mental symptom is an improvement in anger, an improvement in vigor, an improvement in depression, an improvement in friendliness, an improvement in confusion, or an improvement in anxiety.

(5) The composition according to any one of (1) to (4), wherein the QOL is evaluated by an anti-aging QOL common questionnaire.

(6) The composition according to any one of (1) to (5), wherein a daily intake of the equols is 1 mg or greater.

(7) The composition according to any of (1) to (6), wherein the composition is a food or beverage product.

### Effect of the Invention

The present disclosure can at least produce an effect of providing a technique for improving the QOL for a 40-year-old or older postmenopausal woman without menopausal disorder.

### MODE FOR CARRYING OUT THE INVENTION

One embodiment of the present disclosure is a composition for improving QOL of a 40-year-old or older postmenopausal woman without menopausal disorder, comprising equols.

Equols are represented by General Formula (1) below;

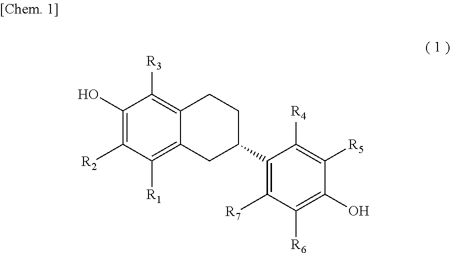

where R₁ to R₇ each represent a hydroxyl group or a hydrogen atom.

Specific examples of the equols include all the compounds included in the compound represented by above General Formula (1), and specific examples thereof include equol (also referred to as 4′,7-isoflavandiol) and 5-hydroxy equol.

The composition for improving QOL according to the present embodiment may contain equols alone, or may contain other components as long as they can improve the QOL of a 40-year-old or older postmenopausal woman without menopausal disorder. Examples of the other components include components such as indigestible dextrin, calcium stearate, and silicon dioxide.

A subject who ingests the composition for improving QOL according to the present embodiment is a 40-year-old or older postmenopausal woman without menopausal disorder. The subject is preferably a person who cannot produce equols.

According to the Japan Society of Obstetrics and Gynecology, the term “menopause” refers to a state in which an ovarian activity is lost gradually and eventually, menstruation stops permanently, and when a woman has not had a menstrual period for 12 months or longer, it is considered the menopause occurred at a date a year ago. The term “postmenopausal” refers to the time of menopause and the time after menopause. Menopause can be confirmed by a commonly used method.

According to the Ministry of Health, Labour and Welfare, the term “menopausal disorder” refers to a syndrome similar to autonomic ataxia due to a decrease in secretion volume of sex hormone. It possible to confirm whether or not the subject has menopausal disorder by a commonly used method. For example, examples of the method include a simplified menopausal index (SMI) score, and with this scoring method, when a subject has a score of 71 or greater, it is confirmed that the subject is experiencing menopausal disorder.

The subject is preferably 40 years old or older, more preferably 45 years old or older, and still more preferably 50 years old or older. Meanwhile, the subject is preferably 80 years old or under, more preferably 75 years old or under, and still more preferably 65 years old or under.

QOL is an abbreviation for “quality of life”, and refers to an extension of healthy life expectancy and quality of life according to the Ministry of Health, Labour and Welfare.

The improvement in QOL in the present embodiment means that QOL is improved when the subject ingests the equols of the present embodiment rather than when the subject does not ingest the equols (or ingests a placebo).

The composition for improving QOL according to the present embodiment may be ingested prophylactically to suppress the reduction in QOL, or may be ingested to maintain QOL.

The evaluation of QOL improvement in the present embodiment may be an objective assessment by a doctor or the like, but the evaluation is preferably performed based on an objective scale using an examination, a questionnaire, or the like developed by an expert.

The QOL improvement in the present embodiment is preferably an improvement in physical symptom or an improvement in mental symptom.

The improvement in physical symptom is preferably an improvement in eye fatigue, an improvement in physical pain (however, eye pain, headache, and stomachache are excluded), an improvement in cardiac ischemia, an improvement in fatigue (however, eye fatigue and feeling of getting eye strain are excluded), an improvement in dry mouth, an improvement in skin condition, an improvement in bronchitis, an improvement in gray hair, an improvement in autonomic nerve balance, an improvement in hearing loss, an improvement in swelling, an improvement in frequent urination, an improvement in insomnia, a maintenance of body weight, a gastric regulation, an improvement of immunity, an intestinal regulation, or hair growth.

The improvement in eye fatigue in the present embodiment is preferably an improvement in eye strain, an improvement in blurred vision, or an improvement in eye pain.

The improvement in physical pain (however, eye pain, headache, and stomachache are excluded) in the present embodiment is preferably an improvement in muscle pain, an improvement in body stiffness (e.g., whole body stiffness, shoulder stiffness, back stiffness, low back stiffness), an improvement in low back pain, or an improvement in joint pain.

The improvement in cardiac ischemia in the present embodiment is preferably an improvement in palpitations or an improvement in breathlessness.

The improvement in fatigue (however, eye fatigue and feeling of getting eye strain are excluded) in the present embodiment is preferably an improvement in physical lethargy or an improvement in feeling of having no sense of well-being.

The improvement in skin condition in the present embodiment is preferably an improvement in skin problems.

Bronchitis in the present embodiment is an improvement in tendency of having a cough, or an improvement in tendency of producing sputum and/or an improvement in difficulty in loosening sputum.

The improvement in autonomic nerve balance in the present embodiment is preferably an improvement in headache, an improvement in dizziness, an improvement in tendency of sweating, an improvement in hot flush, or an improvement in cold constitution.

The improvement in hearing loss in the present embodiment is preferably an improvement in tinnitus or an improvement in difficulty in listening conversation.

The improvement in insomnia in the present embodiment is preferably an improvement in restless sleep, an improvement in difficulty in falling asleep, or an improvement in difficulty in sleeping due to anxiety.

The maintenance of body weight in the present embodiment is preferably an improvement in tendency of gaining weight, an improvement in weight loss, or an improvement in reduced body weight.

The gastric regulation in the present embodiment is preferably an improvement in anorexia, an improvement in stomach tightness, or an improvement in stomachache.

The improvement of immunity in the present embodiment is preferably an improvement in susceptibility to cold viruses.

The intestinal regulation in the present embodiment is preferably an improvement in diarrhea or an improvement in constipation.

The hair growth in the present embodiment is preferably an improvement in hair loss (alopecia).

The improvement in mental symptom is an improvement in anger, an improvement in vigor, an improvement in depression, an improvement in friendliness, an improvement in confusion, or an improvement in anxiety.

The improvement in anger in the present embodiment is preferably an improvement in irritation or an improvement in feeling of being easily angered.

The improvement in vigor in the present embodiment is preferably an improvement in feeling of being unmotivated, an improvement in feeling of being not happy, an improvement in feeling of having no purpose in life, an improvement in feeling of finding no joy in daily life, an improvement in feeling of losing confidence, or an improvement in feeling that oneself is not a useful person.

The improvement in depression in the present embodiment is preferably an improvement in melancholy.

The improvement in friendliness in the present embodiment is preferably an improvement in feeling of being uncomfortable talking with people.

The improvement in confusion in the present implementation is preferably an improvement in feeling of worrying, an improvement in forgetfulness, an improvement in inability to concentrate, an improvement in inability to solve problems, or an improvement in inability to easily judge things.

The improvement in anxiety in the present embodiment is an improvement in tension, an improvement in feeling anxious for no reason, or an improvement in feeling of being afraid.

In a case where the evaluation of QOL improvement in the present embodiment is evaluated by an objective scale such as an examination, a questionnaire, or the like developed by an expert, examples thereof include evaluation by an anti-aging QOL common questionnaire (issued by Japanese Society of Anti-Aing Medicine).

When the evaluation is performed using the anti-aging QOL common questionnaire, it is possible to evaluate the fact that the QOL is improved based on a decrease in the numerical values of respective factors obtained in the evaluation.

The improvement in mental symptom may be evaluated by POMS2. Examples of POMS2 include “Profile of Mood States 2nd Edition-Adult; POMS2” and “Profile of Mood States 2nd Edition-Adult Short; POMS2”. The evaluation by POMS2 can be performed according to the manual of POMS2.

In a case where the improvement in mental symptom is evaluated by POMS2, the mental symptom, anger, vigor, depression, friendliness, confusion, and anxiety correspond to “total mood disturbance”, “anger-hostility”, “vigor-activity”, “depression-dejection”, “friendliness”, “confusion-bewilderment”, and “anxiety-tension” in POMS2, respectively.

When the improvement in mental symptom is evaluated by POMS2, it is possible to evaluate the fact that the mental symptom is improved based on a decrease in the numerical values of the respective factors of “total mood disturbance”, “anger-hostility”, “confusion-bewilderment”, “depression-dejection”, “fatigue-inertia”, and “tension-anxiety” obtained by the evaluation, meanwhile, it is possible to evaluate the fact that the mental symptom is improved based on an increase in the numerical values of the respective factors of “vigor-activity” and “friendliness”.

The content of the equols relative to the total amount of the composition according to the present embodiment is not particularly limited as long as the composition exhibits the action and effect of the equols, but the total amount of the equols is preferably 0.001 mass % or greater, more preferably 0.01 mass % or greater, and still more preferably 0.1 mass % or greater, meanwhile the total amount is preferably 20 mass % or less, more preferably 10 mass % or less, and still more preferably 5 mass % or less.

The intake of the composition according to the present embodiment is appropriately set according to the subject's age and body weight, the ingestion route, the ingestion schedule, the form, and the like, but the ingestion amount is not particularly limited as long as the composition exhibits the action and effect of the equols in the subject through the ingestion of the composition. The total amount of equols is preferably 1 mg or greater, more preferably 2 mg or greater, and still more preferably 5 mg or greater per day, meanwhile the total amount is preferably 40 mg or less, more preferably 20 mg or less, and still more preferably 10 mg or less.

As for the ingestion schedule of the composition of the present embodiment, the composition may be ingested once per day, or may be ingested multiple times per day. Further, the composition may be ingested once in several days or several weeks, but is preferably ingested on a daily basis. Furthermore, the ingestion period is preferably 4 weeks or longer, more preferably 8 weeks or longer. Meanwhile, the upper limit of the ingestion period is not particularly limited, but is for example, a period up to 100 years of age, and may be a period up to death.

The composition according to the present embodiment can be used as a food or beverage product (including a supplement). The composition is, for example, a food or beverage product for improving the QOL of a 40-year-old or older postmenopausal woman without menopausal disorder, comprising equols.

When equols are used as a material for food or beverage products, in addition to general food or beverage products, the equols can also be used as, for example, a food for specified health use, a nutritional supplement, a functional food, a food for a sick person, and a food additive (beverages are also included in these). The form of the food or beverage product may not be necessarily a form of the plant or animal itself containing the equols. For example, the food or beverage product may be prepared by adding an appropriate auxiliary agent to the equols, followed by, using a commonly used means, shaping into a form for consumption such as granules, particles, tablets, capsules, or a paste, and thus supplied for use as food. Alternatively, the food or beverage product may be prepared by adding the equols to various food products, for example, processed meats such as ham and sausage; processed seafoods such as steamed seasoned fish paste (Kamaboko) and tube-shaped fish paste cake (Chikuwa); and breads, confectioneries, butter, powdered milk, and fermented milk products and then used, and may be added to a beverage such as water, fruit juice, milk, or soft drinks and then used.

The food or beverage product can contain, as main ingredients, water, proteins, carbohydrates, lipids, vitamins, minerals, organic acids, organic bases, fruit juices, flavors, and the like. Examples of the proteins include animal and vegetable proteins such as powdered whole milk, powdered skim milk, powdered partially skim milk, casein, soy protein, chicken egg protein, meat protein, hydrolysates thereof and butter. Examples of the carbohydrates include sugars, processed starches (dextrin, as well as soluble starches, British starch, oxidized starch, starch esters, starch ethers, and the like), and dietary fiber. Examples of the lipids include lard, safflower oil, corn oil, rapeseed oil, coconut oil, fractionated oils thereof, hydrogenated oils thereof, transesterified oils, and other such vegetable oils and fats. Examples of vitamins include vitamin A, carotenes, vitamin B group vitamins, vitamin C, vitamin D group vitamins, vitamin E, vitamin K group vitamins, vitamin P, vitamin Q, niacin, nicotinic acid, pantothenic acid, biotin, inositol, choline, and folic acid, and examples of minerals include calcium, potassium, magnesium, sodium, copper, iron, manganese, zinc, selenium, and whey minerals. Examples of organic acids include malic acid, citric acid, lactic acid, and tartaric acid. Two or more types of these components may be used in combination, and synthetic products and/or food and beverage products containing large amounts of these may be used.

The food and beverage products can be produced, for example, by a commonly used method. Furthermore, the blended amount of the equols into a food or beverage product, the blending method, and the blending time can be selected as appropriate. Furthermore, if necessary, the obtained food or beverage product can be sealed in an appropriate container such as a bottle, a bag, a can, a box, or a pack.

For the content of the equols relative to the total amount of the food or beverage product, the intake of the food or beverage product, and the ingestion schedule of the food or beverage product, the descriptions of the content of the equols relative to the total amount of the composition according to the present embodiment, the intake of the composition according to the present embodiment, and the ingestion schedule of the composition according to the present embodiment are used.

In a case where equols are used as a material for food or beverage products, and used for each of the above applications, the equols may be a food or beverage product with a description of each of the applications, or may be a food or beverage product with a description of application that is associated with each of the applications.

For example, the “intestinal regulation” can be described as “normalizing the stomach” or “improving constipation”.

Further, the “improvement in eye fatigue” can be described as “alleviating the feeling of eye fatigue”, “supporting the focus adjustment of the eye”, or the like.

Further, the “improvement in insomnia” can be described as “supporting good quality sleep” or the like.

Further, the “improvement in fatigue” can be described as “reducing mental and physical fatigue” or the like.

Furthermore, the “improvement in vigor” can be described as “making a temporarily depressed mood positive”, “making a temporarily depressed mood active”, “making a temporarily depressed mood lively”, “making a temporarily depressed mood motivated”, and the like.

The equols comprised in the composition for improving QOL according to the present embodiment may be a composition comprising equols, and for example, may be a fermented product of a soy germ extract comprising equols.

Therefore, the composition for improving QOL according to the present embodiment may be a composition for improving QOL of a 40-year-old or older postmenopausal woman without menopausal disorder, comprising equols.

Further, the composition for improving QOL according to the present embodiment may be a composition for improving QOL of a 40-year-old or older postmenopausal woman without menopausal disorder, comprising a fermented product of a soy germ extract comprising equols.

Furthermore, the composition for improving QOL according to the present embodiment may be a composition for improving QOL of a 40-year-old or older postmenopausal woman without menopausal disorder, comprising equols obtained by fermentation of a soy germ extract.

The soy germ extract is not particularly limited as long as it contains equols after being fermented to form a fermented product, and examples thereof include a soy germ extract containing isoflavones. Such a soy germ extract containing isoflavones can be extracted from soybean and soy germ (hypocotyl). Water, ethanol, or hydrous ethanol can be used for the extraction. In addition, for example, the extract can be obtained as in the literature “Development of Materials Containing Plant Polyphenol-its function and safety-” (published by CMC Publishing Co., Ltd. in 2007).

The isoflavones are one of the groups of polyphenols, and are flavonoids with isoflavone as their basic backbone. Isoflavones are contained in a large amount in plants of the family Fabaceae such as soybean, arrowroot (kudzu), red clover, and licorice. Therefore, the soy germ extract comprising isoflavones may be, as an alternative, an extract comprising isoflavones from plants of the family Fabaceae such as arrowroot, red clover or licorice.

In addition, the soy germ extract may be obtained by extracting or purifying the equols of soy germ as long as the soy germ extract contains the equols after being fermented to form a fermented product. Specifically, the soy germ extracts may be isoflavones or a composition containing isoflavones, and may be, for example, a composition containing isoflavones as described in Examples.

The isoflavones are not particularly limited as long as they are fermented into equols, and examples thereof include daidzein, genistein, and glycitein.

In addition, the isoflavones include an isoflavone aglycone and its glycoside, and any derivatives thereof, such as malonylation and acetylation.

Examples of the isoflavone aglycone include daidzein, 6-hydroxydaidzein, dihydroxydaidzein, genistein, glycitein, biochanin A, formononetin, and coumestrol. The isoflavone aglycone may be isoflavones converted by an enzyme such as 3-glucosidase or microorganisms having 3-glucosidase.

Examples of the glycoside include genistin, glycitin, daidzin, and puerarin.

The isoflavones are not necessarily purified isoflavones in the pure form. The type, form, purification degree, and the like of the isoflavones can be arbitrarily selected according to the intended use, application, and the like of the equols obtained by fermentation.

The amount and concentration of the soy germ extract used for fermentation are not particularly limited as long as the soy germ extract contains equols after being fermented to form a fermented product. Further, in a preferred embodiment, when the soy germ extract contains isoflavones, the amount and concentration of the isoflavones used for fermentation are also not particularly limited. Furthermore, in a preferred embodiment, the amount and concentration of the isoflavones used for fermentation in the case of using the isoflavones alone are also not particularly limited. The amount and concentration of the isoflavones can be appropriately set according to the amount of the culture solution and the amount of equols to be produced.

In fermentation, anaerobic microorganisms are cultured in a culture medium containing a soy germ extract. The aerobic microorganisms are not particularly limited as long as they are anaerobic microorganisms capable of producing equols at a temperature of about 37° C. (e.g., 30 to 42° C.), and examples thereof include microorganisms belonging to the genus Adlercreutzia, microorganisms belonging to the genus Asaccharobacter, microorganisms belonging to the genus Bacteroides, microorganisms belonging to the genus Bifidobacterium, microorganisms belonging to the genus Eggerthella, microorganisms belonging to the genus Enterococcus, microorganisms belonging to the genus Eubacterium, microorganisms belonging to the genus Lactobacillus, microorganisms belonging to the genus Lactococcus, microorganisms belonging to the genus Sharpea, microorganisms belonging to the genus Slackia, and microorganisms belonging to the genus Streptococcus. Among them, microorganisms belonging to the genus Asaccharobacter and microorganisms belonging to the genus Adlercreutzia are preferred.

In addition, examples of the microorganisms belonging to the genus Adlercreutzia include microorganisms belonging to Adlercreutzia equolifaciens and microorganisms belonging to Adlercreutzia mucosicola; examples of the microorganisms belonging to the genus Asaccharobacter include microorganisms belonging to Asaccharobacter celatus; microorganisms belonging to the genus Bacteroides include microorganisms belonging to Bacteroides ovatus; examples of the microorganisms belonging to the genus Bifidobacterium include microorganisms belonging to Bifidobacterium animal is, microorganisms belonging to Bifidobacterium breve, microorganisms belonging to Bifidobacterium longum, and microorganisms belonging to Bifidobacterium pseudolongum; examples of the microorganisms belonging to the genus Enterococcus include microorganisms belonging to Enterococcus faecalis; examples of the microorganisms belonging to the genus Lactobacillus include microorganisms belonging to Lactobacillus fermentum, microorganisms belonging to Lactobacillus intestinalis, microorganisms belonging to Lactobacillus plantarum, and microorganisms belonging to Lactobacillus rhamnosus; examples of the microorganisms belonging to the genus Lactococcus include microorganisms belonging to Lactococcus garvieae; examples of the microorganisms belonging to the genus Sharpea include microorganisms belonging to Sharpea azabuensis; examples of the microorganisms belonging to the genus Slackia include microorganisms belonging to Slackia equolifaciens and microorganisms belonging to Slackia isoflavoniconvertens; and examples of the microorganisms belonging to the genus Streptococcus include microorganisms belonging to Streptococcus constellatus and microorganisms belonging to Streptococcus intermedius.

In addition, examples of the microorganisms belonging to Adlercreutzia equolifaciens include the Adlercreutzia equolifaciens FJC-B9T strain; examples of the microorganisms belonging to Adlercreutzia mucosicola include Adlercreutzia mucosicola Mut1B8 strain; examples of the microorganisms belonging to Asaccharobacter celatus include Asaccharobacter celatus AHU 1763 strain and Asaccharobacter celatus DSM 18785 strain; examples of the microorganisms belonging to Bacteroides ovatus include Bacteroides ovatus E-23-15 strain; examples of the microorganisms belonging to Bifidobacterium breve include Bifidobacterium breve ATCC 15700 strain; examples of the microorganisms belonging to Bifidobacterium longum include Bifidobacterium longum BB 536 strain; examples of microorganisms belonging to the genus Eggerthella include Eggerthella sp. YY7918 strain and Eggerthella sp. D1 strain; examples of the microorganisms belonging to Enterococcus faecalis include Enterococcus faecalis INIA P 333 strain; examples of the microorganisms belonging to the genus Eubacterium include Eubacterium sp. D2 strain; examples of the microorganisms belonging to Lactobacillus fermentum include Lactobacillus fermentum DPPMA 114 strain; examples of the microorganisms belonging to Lactobacillus intestinalis include Lactobacillus intestinalis KCT13676 BP strain; examples of the microorganisms belonging to Lactobacillus plantarum include Lactobacillus plantarum DPPMA24W strain and Lactobacillus plantarum DPPMASL 33 strain; examples of the microorganisms belonging to Lactobacillus rhamnosus include Lactobacillus rhamnosus INIA P540 strain and Lactobacillus rhamnosus DPPMAAZ1; examples of the microorganisms belonging to Lactococcus garvieae include Lactococcus garvieae 20-92 strain; examples of the microorganisms belonging to Sharpea azabuensis include Sharpea azabuensis ST18 strain; examples of the microorganisms belonging to Slackia equolifaciens include Slackia equolifaciens DZE strain; examples of the microorganisms belonging to Slackia isoflavoniconvertens include Slackia isoflavoniconvertens HE8 strain; examples of the microorganisms belonging to the genus Slackia include Slackia sp. TM-30 strain, Slackia sp. FJK1 strain, Slackia sp. NATTS strain, and Slackia sp. YIT 11861 strain; examples of the microorganisms belonging to Streptococcus constellatus include Streptococcus constellatus E-23-17 strain; and examples of the microorganisms belonging to Streptococcus intermedius include Streptococcus intermedius A6G-225 strain.

The anaerobic microorganisms are cultured in a culture medium containing a soy germ extract under conditions suitable for the production of equols. The conditions suitable for the production of equols refer to conditions under which the survival and activity of the anaerobic microorganisms having the activity of producing equols are maintained. More specifically, the conditions refer to conditions under which the gas phase conditions (anaerobic conditions) in which the anaerobic microorganisms can survive are maintained, and nutrients for supporting the activity and growth of the anaerobic microorganisms are provided. Various medium compositions suitable for the survival of the anaerobic microorganisms are known. Therefore, in the case of using the anaerobic microorganisms, those skilled in the art can select an appropriate medium composition. For example, a BHI culture medium manufactured by Difco Laboratories, a GAM broth medium manufactured by NISSUI PHARMACEUTICAL CO., LTD. used in Examples, and the like can be used.

For example, a water soluble organic material can be added, as a carbon source, to the culture medium. Examples of the water-soluble organic material include saccharides such as sorbose, fructose, and glucose; alcohols such as methanol; and organic acids such as valeric acid, butyric acid, propionic acid, acetic acid, and formic acid.

The concentration of organic material added in the culture medium as a carbon source can be adjusted, as appropriate, to efficiently grow the anaerobic microorganisms in the culture medium. In general, the addition amount is selected from the range of 0.1 to 10 wt/vol %, and thus it is possible to avoid excess or deficiency in the addition amount.

In addition to the carbon source described above, a nitrogen source is added to the culture medium. Various nitrogen compounds that may be used ordinarily in fermentation can be used as the nitrogen source. Preferred inorganic nitrogen sources are ammonium salts and nitrates. Examples of more preferred inorganic nitrogen sources include ammonium sulfate, ammonium chloride, ammonium phosphate, ammonium hydrogenphosphate, potassium nitrate, and sodium nitrate.

Meanwhile, examples of preferred organic nitrogen sources include amino acids, yeast extracts, peptones (e.g., polypeptone N), meat extracts, liver extracts, and digested serum powder. Examples of more preferred organic nitrogen sources include arginine, cysteine, citrulline, lysine, yeast extracts, and peptones (e.g., polypeptone N).

In addition, other organic materials or inorganic materials suitable for culturing the anaerobic microorganisms can also be added to the culture medium, in addition to the carbon source and the nitrogen source. For example, in some cases, the growth and activity of the anaerobic microorganisms can be enhanced by adding cofactors such as vitamins or inorganic compounds such as various salts to the culture medium. Examples of plant- and animal-derived cofactors for microbial growth, such as inorganic compounds and vitamins, include the following.

| Inorganic Compounds | Vitamins |
|---|---|
| Potassium dihydrogen phosphate | Biotin |
| Magnesium sulfate | Folic acid |
| Manganese sulfate | Pyridoxine |
| Sodium chloride | Thiamine |
| Cobalt chloride | Riboflavin |
| Calcium chloride | Nicotinic acid |
| Zinc sulfate | Pantothenic acid |
| Copper sulfate | Vitamin B12 |
| Potassium alum | Thioctic acid |
| Sodium molybdate | p-aminobenzoic acid |
| Potassium chloride | |
| Boric acid, etc. | |
| Nickel chloride | |
| Sodium tungstate | |
| Sodium selenate | |
| Iron (II) ammonium sulfate | |

Methods for producing a culture solution by adding these inorganic compounds and vitamins, or growth cofactors are well known. The culture medium can be a liquid, a semi-solid, or a solid. A preferred form of the culture medium is a liquid culture medium.

The anaerobic microorganisms can be cultured according to a known method of culturing anaerobic microorganisms. In industrial production, a continuous cultivation system (continuous fermentation system) that is capable of continuously feeding the culture medium and gaseous substrate and is provided with a mechanism for recovering the culture is preferred.

In culturing anaerobic microorganisms, it is necessary to prevent oxygen from mixing into the continuous cultivation system. As an incubator, a commonly used culture vessel can be used directly. Culture vessels that can be used in the cultivation of the anaerobic microorganisms are commercially available. An anaerobic atmosphere can be created by replacing oxygen that mixes into the culture vessel with an inert gas such as nitrogen or a gaseous substrate.

As the culture vessel, a culture tank provided with an additional function can be used. For example, in addition to an ordinarily used stirring and mixing vessel, a bubble column type or a draft tube type culture vessel can also be used. The anaerobic microorganisms are freely dispersed by circulation of a mixed gas into the liquid culture medium, and contact between the anaerobic microorganisms and the culture medium can be sufficiently obtained. Alternatively, the anaerobic microorganisms can also be inhabited with drops of water in the filling layer of a slag having high permeability such as a biotrickling filter, other inorganic ceramic filler, or synthetic organic substances such as polypropylene, to be cultured while the gas is passed through. Furthermore, the anaerobic microorganisms that are used can be immobilized in a carrageenan gel, alginate gel, acrylamide gel, chitin, cellulose, agar, and the like by a commonly used method.

Depending on the shape of the culture vessel, a stirrer or the like can be used to sufficiently stir the culture medium. The production efficiency of equols can be optimized by stirring the culture in the culture vessel to thereby increase the opportunities for contact of the components of the culture medium and the gaseous substrate with anaerobic microorganisms. The gaseous substrate can also be supplied as nanobubbles.

Due to sufficient growth of the anaerobic microorganisms, the pH of the culture is preferably from 5.0 to 8.0, more preferably from 6.0 to 7.5, and still more preferably from 6.5 to 7.5. Further, the temperature of the culture vessel is not particularly limited, but is preferably from 30° C. to 40° C., and more preferably from 33° C. to 38° C. since the recovery amount of the equols can be increased.

The fermentation time can be appropriately set according to the production amount of equols, the remaining amount of soy germ extract, and the like. The fermentation time is, for example, from 8 to 120 hours, preferably from 12 to 72 hours, and particularly preferably from 16 to 60 hours.

The anaerobic microorganisms are preferably cultured in a gas phase consist of one or more gases containing hydrogen. The hydrogen concentration is not particularly limited.

The resulting culture solution may be subjected to solid-liquid separation by filtration or centrifugal separation or the like, and the liquid phase may be recovered. Examples of the filtration include filtration using a filter paper or ultrafiltration membrane, and examples of the centrifugal separation include centrifugal separation using a centrifuge such as a super decanter. In addition, the resulting culture solution may be used as it is without solid-liquid separation.

For the fermented product of the soy germ extract, the resulting culture solution is formed into a solid by a process of heat-drying, spray-drying, or freeze-drying, as necessary, and the resultant solid may be used. The heat-drying process and the spray-drying process can both be performed using, for example, a spray-drying apparatus. The freeze-drying process can be performed using a freeze-drying apparatus.

The heat-dried, spray-dried, or freeze-dried fermented product may be subjected to a pulverizing process as necessary.

The heat-dried, spray-dried, or freeze-dried fermented product obtained by such a method can include equols in an amount of 0.05 to 50 mass % in the dry powder. For example, the content is preferably expressed in mass % of a concentration range in which a pair of concentrations in mass % selected from 0.1%, 0.5%, 1%, 2%, 3%, 4%, 5%, 6%, 7%, 8%, 9%, 10%, 20%, 30%, 40%, and 50% are used as the lower limit (X or grater, or, greater than X) and the upper limit (Y or less, or, less than Y), respectively.

### EXAMPLES

Examples are described below, but none of the examples are interpreted to be limiting.

### (Production Example 1) Production of Composition for Improving QOL

As raw materials, a fermented product of a soy germ extract (84 mg) containing equol (5 mg), 136.8 mg of indigestible dextrin, 6.9 mg of calcium stearate, and 2.3 mg of silicon dioxide were filled into HPMC capsules to produce a composition for improving QOL. This composition was used as a test meal in the test described later.

The fermented product of the soy germ extract was prepared as follows.

First, a seed culture solution of anaerobic microorganisms used for fermentation was prepared. Specifically, 5.9 g of GAM broth medium (manufactured by NISSUI PHARMACEUTICAL CO., LTD.) and 1.2 g of L-arginine hydrochloride were dissolved in 100 mL of pure water, and each 10 mL was dispensed into an 18 mm test tube for culturing anaerobic bacteria (manufactured by SANSHIN INDUSTRIAL CO., LTD.) while passing nitrogen gas. The test tube was closed up with a butyl rubber stopper and a plastic cap, followed by sterilization at 115° C. for 15 minutes. Asaccharobacter celatus DSM 18785 strain, which Anaerobic microorganisms that had been cryopreserved at −80° C., was inoculated into this culture medium, and the gas phase was replaced for 2 minutes or more with hydrogen gas passed through a sterile filter (percent concentration of hydrogen partial pressure: 100%). Thereafter, the resultant culture was subjected to shaking culture at 37° C. and 250 spm for 18 hours to prepare a seed culture solution.

Next, a culture medium was prepared containing 11 g of isoflavone-80 (manufactured by J-OIL MILLS, INC.), 3 g of yeast extract, 0.5 g of L-cysteine hydrochloride, 0.002 g of hemin, 16.5 g of β-cyclodextrin, 12.1 g of L-arginine hydrochloride, 0.1 g of oleic acid, and 0.2 g of an antifoaming agent, adjusted to 1000 mL with deionized water, and adjusted to pH 7.1. 1000 mL of the culture medium was put into a 2 L volume pressurized mini jar (manufactured by Automatic system Research. Co., Ltd.), and sterilized at 115° C. for 15 minutes after passing nitrogen gas for 20 minutes or more. The isoflavone-80 (manufactured by J-OIL MILLS, INC.) is a powder containing high-purity soybean isoflavone glycosides (daidzin, glycitin, and genistin) obtained by extracting and purifying a raw material: soy germ from North America in Japan (Soybean functional material (Fine), [online], J-OIL MILLS, INC. [searched on May 14, 2020], on the Internet at http://www.j-oil.com/product/gyoumu/fine/>). In this test, the glycosides were hydrolyzed in advance using a commercially available 3-glucosidase to form aglycones (daidzein, glycitein, and genistein, respectively).

20 mL of the seed culture solution was inoculated into this culture medium, a gas of hydrogen/nitrogen [2: 98] (volume ratio) was aseptically aerated at 0.1 L/min from a sparger, and cultivation was performed at 37° C., 500 rpm, and 50 kPa. The pH during culture was adjusted to 7.2 with sulfuric acid. After 48 hours, the culture solution was taken out.

The culture solution was adjusted to pH 5.0 with sulfuric acid, and then heat-treated at 105° C. for 1 minute. The resultant solution was cooled to room temperature, and then subjected to centrifugal separation at 15000×g for 10 minutes at 20° C. The supernatant fluid was spray-dried to give a fermented product. The equol content was 6.5%. In addition, the components other than equol in the fermented product were 43 g of protein, 3 g of lipid, 50 g of carbohydrate, 50 mg of sodium, and 2 g of ash, per 100 g. The calorie of the fermented product was 400 kcal per 100 g.

### Example 1

30 healthy women aged between 40 and under 65 who did not produce equol and were postmenopausal without menopausal disorder (simplified menopausal index is 70 points or less) were allowed to ingest one test meal with a glass of water or warm water twice a day for 12 weeks. Before ingestion and 8 weeks after ingestion, QOL was evaluated using an anti-aging QOL common questionnaire.

The evaluation was performed in accordance with the manual of the anti-aging QOL common questionnaire. Specifically, the women were allowed to answer in a scale of five levels: “1. None at all”, “2. Almost none”, “3. Some”, “4. Moderate”, and “5. High”, and “1.” was scored as 1 point and “5.” was scored as 5 point.

The test results were treated as non-parametric data and comparisons between pre-ingestion and 8 weeks after ingestion were performed using Wilcoxon signed rank test and determined with a two-tailed test.

Results

The results are shown in Tables 1 to 3. The numerical values indicate the mean±standard deviation. It was found that the composition for improving QOL according to the present example improved the QOL of subjects.

TABLE 1

| | | 8 weeks | |
|---|---|---|---|
| | Before | after | |
| | ingestion | ingestion | P-value |
| QOL | 144.8 ± 21.7 | 127.5 ± 25.2 | P < 0.01 |
| Physical symptom | 91.0 ± 13.6 | 80.9 ± 15.4 | P < 0.01 |
| Mental symptom | 53.8 ± 11.6 | 46.6 ± 11.9 | P < 0.01 |

TABLE 2

| | | | 8 weeks | |
|---|---|---|---|---|
| | | Before | after | |
| | | ingestion | ingestion | P-value |
| Eye fatigue | Getting eye strain | 3.6 ± 0.8 | 3.2 ± 0.9 | P < 0.05 |
| | Getting blurred vision | 3.3 ± 1.0 | 3.0 ± 0.9 | |
| | Eye pain | 2.5 ± 1.2 | 2.1 ± 0.9 | |
| Physical pain | Shoulder stiffness | 4.3 ± 0.8 | 3.8 ± 1.1 | P < 0.01 |
| | Muscle pain and stiffness | 3.6 ± 1.2 | 3.0 ± 0.9 | P < 0.05 |
| | Low back pain | 3.1 ± 1.1 | 2.9 ± 1.2 | |
| | Joint pain | 2.7 ± 1.1 | 2.3 ± 1.2 | |
| Cardiac | Palpitation | 2.2 ± 0.9 | 2.1 ± 0.9 | |
| ischemia | Breathlessness | 2.5 ± 1.0 | 2.3 ± 1.0 | |
| Fatigue | Feeling of weariness | 3.1 ± 0.7 | 2.7 ± 0.8 | P < 0.01 |
| | Feeling of having no sense of well- | 2.4 ± 0.6 | 2.2 ± 0.7 | |
| | being | | | |
| Dry mouth | Dry mouth | 2.9 ± 1.0 | 2.6 ± 0.9 | P < 0.05 |
| Skin condition | Skin problems | 2.8 ± 0.8 | 2.4 ± 0.8 | P < 0.05 |
| Bronchitis | Cough or sputum | 2.3 ± 0.9 | 2.1 ± 0.8 | |
| Gray hair | Gray hair | 4.0 ± 0.9 | 3.6 ± 0.9 | P < 0.01 |
| Autonomic | Headache | 2.7 ± 1.1 | 2.4 ± 1.0 | |
| nerve balance | Dizziness | 2.2 ± 0.9 | 2.0 ± 0.8 | |
| | Tendency of sweating | 3.3 ± 1.0 | 2.8 ± 1.0 | P < 0.05 |
| | Hot flush | 3.2 ± 1.0 | 2.6 ± 1.1 | P < 0.01 |
| | Cold constitution | 3.6 ± 1.1 | 2.9 ± 1.0 | P < 0.01 |
| Hearing loss | Tinnitus | 2.2 ± 1.2 | 1.7 ± 0.9 | P < 0.01 |
| | Difficulty in listening conversation | 2.5 ± 1.2 | 2.3 ± 1.2 | |
| Swelling | Swelling | 2.8 ± 0.9 | 2.4 ± 1.0 | P < 0.05 |
| Frequent | Frequent urination | 2.9 ± 1.3 | 2.8 ± 1.2 | |
| urination | | | | |
| Insomnia | Restless sleep | 3.2 ± 1.0 | 2.9 ± 1.1 | P < 0.05 |
| | Difficulty in falling asleep | 2.9 ± 1.1 | 2.6 ± 1.2 | |
| | Difficulty in sleeping due to anxiety | 2.7 ± 0.9 | 2.3 ± 0.8 | P < 0.05 |
| Maintenance | Tendency of gaining weight | 3.3 ± 0.9 | 3.1 ± 1.0 | |
| of body weight | Weight loss/reduced body weight | 1.6 ± 0.9 | 1.4 ± 0.6 | |
| Gastric | Anorexia | 1.9 ± 0.6 | 1.6 ± 0.7 | P < 0.01 |
| regulation | Stomach tightness | 2.4 ± 1.0 | 2.0 ± 0.8 | P < 0.01 |
| | Stomachache | 2.0 ± 1.0 | 1.9 ± 0.9 | |
| Improvement | Susceptibility to cold viruses | 2.0 ± 0.6 | 1.9 ± 0.7 | |
| of immunity | | | | |
| Intestinal | Diarrhea | 2.2 ± 1.1 | 2.0 ± 0.9 | |
| regulation | Constipation | 2.2 ± 1.0 | 2.2 ± 1.1 | |
| Hair growth | Hair loss | 2.9 ± 0.8 | 2.7 ± 1.0 | |

TABLE 3

| | | | 8 weeks | |
|---|---|---|---|---|
| | | Before | after | |
| | | ingestion | ingestion | P-value |
| Anger | Getting irritable | 2.9 ± 0.6 | 2.4 ± 0.8 | P < 0.01 |
| | Being easily angered | 2.8 ± 0.7 | 2.3 ± 0.8 | P < 0.01 |
| Vigor | Being unmotivated | 2.7 ± 0.8 | 2.1 ± 0.9 | P < 0.01 |
| | Feeling of being not happy | 2.2 ± 0.7 | 1.9 ± 0.7 | P < 0.01 |
| | Feeling of having no purpose in life | 2.1 ± 0.8 | 1.8 ± 0.8 | P < 0.05 |
| | Finding no joy in daily life | 2.2 ± 0.7 | 1.8 ± 0.7 | P < 0.01 |
| | Losing confidence | 2.4 ± 1.0 | 2.0 ± 0.7 | P < 0.01 |
| | Feeling of being not a useful person | 2.1 ± 0.8 | 1.9 ± 0.7 | |
| Depression | Melancholy | 2.1 ± 0.7 | 1.9 ± 0.7 | |
| Friendliness | Being uncomfortable talking with | 2.0 ± 0.8 | 1.8 ± 0.7 | |
| | people | | | |
| Confusion | Worrying | 2.5 ± 0.8 | 2.2 ± 0.7 | |
| | Getting forgetful | 3.5 ± 1.1 | 3.3 ± 1.0 | |
| | Inability to concentrate | 3.0 ± 0.8 | 2.6 ± 0.9 | P < 0.01 |
| | Inability to solve problems | 2.4 ± 0.7 | 2.3 ± 0.8 | |
| | Inability to easily judge | 2.6 ± 0.8 | 2.1 ± 0.8 | P < 0.01 |
| Anxiety | Tension | 2.7 ± 0.7 | 2.3 ± 0.8 | P < 0.05 |
| | Feeling anxious for no reason | 2.5 ± 0.9 | 2.1 ± 0.8 | P < 0.01 |
| | Feeling of being afraid | 2.2 ± 1.0 | 1.8 ± 0.8 | P < 0.05 |

1. A method for improving QOL of a 40-year-old or older postmenopausal woman without menopausal disorder, comprising administering a composition comprising equols to said woman, in an effective amount for improving QOL of said woman.

2. The method according to claim 1, wherein the QOL improvement is an improvement in physical symptom or an improvement in mental symptom.

3. The method according to claim 2, wherein the improvement in physical symptom is an improvement in eye fatigue, an improvement in physical pain, an improvement in cardiac ischemia, an improvement in fatigue, an improvement in dry mouth, an improvement in skin condition, an improvement in bronchitis, an improvement in gray hair, an improvement in autonomic nerve balance, an improvement in hearing loss, an improvement in swelling, an improvement in frequent urination, an improvement in insomnia, a maintenance of body weight, a gastric regulation, an improvement of immunity, an intestinal regulation, or hair growth.

4. The method according to claim 2, wherein the improvement in mental symptom is an improvement in anger, an improvement in vigor, an improvement in depression, an improvement in friendliness, an improvement in confusion, or an improvement in anxiety.

5. The method according to claim 1, wherein the QOL is evaluated by an anti-aging QOL common questionnaire.

6. The method according to claim 1, wherein the equols are administered to the woman at a daily dose of 1 mg or greater.

7. The method according to claim 1, wherein the composition is a food or beverage product.
